(19) United States
(12) Patent Application Publication
(10) Pub. No.: US 2011/0190794 A1
(43) Pub. Date: Aug. 4, 2011

(54) MEDICAL NEEDLE HOLDER

(75) Inventors: Andreas DINGLER, Birkenfeld (DE); Ernst FALK, Sternenfels-Diefenbach (DE); Manfred BOEBEL, Bauschlott (DE)

(73) Assignee: RICHARD WOLF GMBH, Knittlingen (DE)

(21) Appl. No.: 13/015,662

(22) Filed: Jan. 28, 2011

(30) Foreign Application Priority Data
Jan. 29, 2010 (DE) 10 2010 006 202.2

(51) Int. Cl. A61B 17/062

(52) U.S. Cl. 606/147

(57) ABSTRACT

A medical needle holder is provided having a handle arranged at the proximal end and a needle receiver arranged at the distal end. The needle receiver has a first and a second clamping surface, which clamping surfaces face toward each other and extend in a direction transverse to the longitudinal axis of the holder. The clamping surfaces are movable relative to each other via the handle, in the direction of the longitudinal axis of the holder.

### BACKGROUND OF THE INVENTION

The invention relates to a medical needle holder having a handle arranged at the proximal end and a needle receiver arranged at the distal end.

Particularly in endoscopic operations, needle holders are used to hold or guide needles when suturing wounds. In the known needle holders, the needles can be gripped only in a certain position. In addition, a lock is generally provided, which first has to be undone before the needle is released. This makes handling awkward.

### BRIEF SUMMARY OF THE INVENTION

An object of the invention is to make available an improved medical needle holder that is easier to handle.

This object is achieved by a medical needle holder of the type mentioned at the outset, wherein the needle receiver has a first and a second clamping surface, which clamping surfaces face toward each other, and extend in a direction transverse to the longitudinal axis of the instrument and are movable relative to each other, via the handle, in the direction of the longitudinal axis of the instrument. Preferred embodiments will become clear from the present description as well as from the figures.

The medical needle holder according to the invention comprises, in a known manner, a handle at the proximal end and a needle receiver at the distal end, in which needle receiver a needle can be held. The handle and needle receiver are connected to each other by an instrument shaft. By actuating the handle, it is possible to open and close the needle receiver, such that needles can be released and held, respectively.

According to the invention, the needle receiver comprises a first and a second clamping surface, between which clamping surfaces a needle can be held or clamped. For this purpose, the first and the second clamping surface are arranged in such a way that they face toward each other and lie opposite each other. According to the invention, the first and the second clamping surface extend in a direction transverse to the longitudinal axis of the instrument, i.e. the surfaces extend at an angle to the longitudinal axis, preferably perpendicularly, i.e. at an angle of 90 degrees to the longitudinal axis of the instrument. Other angles are also conceivable, however, for example between 45 and 90 degrees. Moreover, the two clamping surfaces are movable relative to each other, via the handle, in the direction of the longitudinal axis of the instrument. That is to say, the clamping surfaces are moved toward each other or away from each other in the direction of the longitudinal axis of the instrument in order to close or open the clamping area between the surfaces, depending on the direction of movement. In an angled configuration of the instrument, the movement does not necessarily have to take place in the direction of the longitudinal axis of the instrument, i.e. in the direction of the longitudinal axis of the instrument shaft, but instead can take place in the direction of a longitudinal axis of the needle receiver, which is at an angle to the longitudinal axis of the instrument. However, the longitudinal axis is always the axis that extends from the proximal to the distal end of the instrument.

The design of the needle receiver according to the invention has the advantage that needles can be gripped in different positions, since in this way clamping areas between the clamping surfaces can be made available on several sides of the longitudinal axis of the instrument.

For this purpose, it is furthermore preferable that the first and second clamping surface are each ring-shaped, wherein a gap formed between the clamping surfaces is open toward its outer circumference. The ring-shaped clamping surfaces extend about the longitudinal axis of the instrument or the longitudinal axis of the needle receiver, along which axis the clamping surfaces are moved relative to each other. The first and second clamping surfaces preferably extend concentrically to this longitudinal axis. The gap between the clamping surfaces forms the clamping area, which can be closed by the two clamping surfaces being moved toward each other and bearing on each other, and which can be opened by the two clamping surfaces being moved away from each other. Since this gap is open toward the outer circumference, needles can be received from all sides of the longitudinal axis. To this extent, the orientation of the needle holder relative to the needles is simplified, or it is easier to grip a needle, since the needle holder does not necessarily have to be moved into a predetermined position relative to the needle, but instead there are several positions in which needles can be gripped without having to change the position of the instrument.

The clamping surfaces particularly preferably extend in a circular shape about the longitudinal axis of the instrument. A symmetrical structure of the needle receiver is thus achieved, as a result of which handling is further simplified, since there is no need to ensure a defined angle position of the needle receiver.

It is also preferable that the first clamping surface is arranged fixedly at the distal end of an instrument shaft, and the second clamping surface is arranged at the distal end of an actuating rod that is axially movable relative to the shaft. In this way, by moving the actuating rod, it is possible to move the second clamping surface toward the first clamping surface, such that the two clamping surfaces bear on each other, or such that a needle can be clamped between the two clamping surfaces. By moving the actuating rod in the opposite direction, it is possible to move the clamping surfaces away from each other, such that the gap between the first clamping surface and second clamping surface is widened, in order to release or receive a needle. The actuating rod is preferably movable in its axial direction and extends along the longitudinal axis of the instrument in the interior of the instrument shaft.

The first clamping surface preferably faces in the distal direction, and the second clamping surface preferably faces in the proximal direction. The first clamping surface can thus be formed as a radial projection at the distal end of the instrument shaft, and the second clamping surface can be formed as an opposite mating surface that faces toward the proximal side of the instrument.

The second clamping surface preferably projects from a pin that extends through an opening in the first clamping surface. In this way, a mushroom-shaped needle receiver is created, with an annular receiving gap arranged between the clamping surfaces and open toward the outer circumference. The pin is preferably arranged centrally on the second clamping surface and extends through a central opening in the first clamping surface. The pin and the opening are preferably designed concentrically to the longitudinal axis of the instrument. The pin, on which the second clamping surface is formed, is preferably secured on the distal end of an actuating rod or is formed directly by the distal end of the actuating rod. In this way, an instrument structure with a small number of individual parts is achieved, which permits easy assembly and good cleaning of the instrument.

Moreover, the first and/or the second clamping surface expediently have/has a structured surface, in particular a surface provided with staggered teeth. The structuring or teeth of the surface ensures a secure hold of a received needle. These surfaces additionally have the effect that, in addition to a force fit when the needle is clamped, a certain degree of form fit is achieved, which prevents the needle from slipping out in the radial direction from the clamping area between the first and second clamping surface.

Moreover, a pre-tensioning element is expediently provided, by which the first and second clamping surfaces are kept bearing on each other by application of a force. This state is preferably the rest position of the instrument. This ensures that in the rest position, i.e. when the handle is not actuated, a needle can be held securely between the first and second clamping surfaces simply by the pre-tensioning, without the need for an additional manually actuatable lock that prevents opening of the instrument. The pre-tensioning element is preferably a spring which, for example, can be arranged in the area of the handle in order to press the grip members of the handle into their rest position by application of a force. By at least one grip member being mechanically coupled to an actuating rod, which can move the first and second clamping surfaces relative to each other, such a spring element is also able to keep the first and second clamping surfaces bearing on each other.

The medical needle holder is preferably designed in such a way that when the handle is actuated, i.e. when two grip members of the handle are manually pressed together for example, the first and second clamping surfaces can be moved away from each other counter to the pre-tensioning force, in order to open the clamping gap. As has been described, the movement of the handle is preferably transferred by an actuating rod to one of the clamping surfaces or to both clamping surfaces.

According to another preferred embodiment, the needle holder according to the invention has a handle having at least one grip member which, at its distal end, is mounted in an articulated manner and, at its proximal end, is connected to a leaf spring element which, starting from the proximal end of the grip member, extends in the distal direction and, at its distal end away from the grip member, is connected to the proximal end of an actuating rod. The leaf spring element lies in the inside of the handle and is preferably mounted fixedly on the proximal end of the grip member and extends from there in the distal direction in such a way that, along its extent, it moves increasingly farther away from the grip member. At its distal end, the leaf spring element is then connected directly or indirectly to the proximal end of the actuating rod. The leaf spring element is preferably connected to the proximal end of the actuating rod in an articulated manner. The leaf spring element performs two functions. First, it transfers the movement of the grip member to the actuating rod, such that the actuating rod is moved linearly, i.e. in the direction of the longitudinal axis of the instrument, when the grip member is pivoted. During the inward pivoting of the grip member, i.e. toward the longitudinal axis of the instrument, the spring element is preferably moved and deformed in such a way that the point of connection to the actuating rod is moved distally and, consequently, the actuating rod is also moved in the distal direction. Second, the spring element at the same time performs the function of a restoring spring. To move the actuating rod, the leaf spring element is elastically bent or deformed upon pivoting of the grip member. On account of this elastic deformation of the leaf spring element, a restoring force is generated which, when the pressure on the grip member is released, moves the grip member back to its starting position, in which case the actuating rod is also simultaneously moved back to its starting position.

The handle preferably has two such grip members, each one having such a leaf spring element. The leaf spring elements lie in the inside of the handle between the two grip members. The two grip members are mounted in an articulated manner at a common point of attachment or at separate points of attachment. In the latter case, the points of attachment are preferably symmetrical with respect to the longitudinal axis of the instrument, and, in particular, the entire handle is preferably mirror-symmetrical with respect to the longitudinal axis of the instrument. Accordingly, in this embodiment, two leaf spring elements are also provided, which are connected in an articulated manner, preferably at a common point of attachment, to the proximal end of the actuating rod. This point of attachment preferably lies on the line of symmetry of the handle, i.e. preferably on the longitudinal axis of the instrument. The leaf spring elements are curved in their rest position such that, starting from the proximal end spaced apart from the longitudinal axis of the instrument, they curve in toward the longitudinal axis of the instrument and, in so doing, move farther away from the inner side of the associated grip member. When the grip members are moved toward each other, this leads to a deformation of the leaf spring elements, such that the latter adopt a flatter configuration, as a result of which the point of attachment on the actuating rod shifts in the distal direction.

According to another preferred embodiment, the medical needle holder according to the invention additionally has a thread cutter arranged in the area of the distal end and actuatable by the handle. This thread cutter is preferably arranged not quite at the distal end, but proximally behind the needle receiver, yet close to the latter. This configuration has the advantage that needles can be held and also threads can be cut using one and the same instrument, such that additional scissors for cutting the threads are not needed during the operation. The thread cutter is preferably also able to be actuated using the same handle with which the clamping surfaces of the needle receiver are moved, such that a simple instrument structure and also simple handling of the instrument are still ensured.

In order to form the thread cutter, the instrument shaft preferably has, on its circumference, a recess that extends in the radial direction all the way through the shaft wall, wherein a cutting edge is arranged or formed on an axially movable actuating rod in the interior of the instrument shaft in such a way that, upon movement of the actuating rod, this cutting edge travels past the recess. In this way, the cutting edge can cut off or shear off a thread that has been inserted into the recess, and that extends through the recess preferably in the manner of a chord, by the cutting edge moving past the recess. The recess is preferably designed as a slit in the wall of the instrument shaft, the slit preferably extending about such a circumferential area of the instrument shaft that a straight line joining the ends of the slit is located as a chord in the interior of the instrument shaft. That is to say, a thread that has been inserted into the slit can extend in the direction of a chord through the interior of the instrument shaft, such that it can then be shorn off by the cutting edge moving in the interior. The cutting edge is preferably designed such that it travels past the recess or slit only at one circumferential end, or it extends obliquely in such a way that it travels past one end of the slit first. In this way, it is possible to ensure that the thread is not cut twice and a short piece of thread remains in the interior of the instrument shaft.

The cutting edge is preferably formed directly in the actuating rod, such that the number of individual parts is reduced. Alternatively, the cutting edge can also be formed in or on a pin that carries the second clamping surface, in which case this pin is mounted at the distal end of an actuating rod. The part carrying the cutting edge is preferably guided substantially free of play in the interior of the instrument shaft, such that the cutting edge is guaranteed to bear on the inner face in the area of the recess in order to ensure a shearing action for severing a thread. For this purpose, in addition to the cutting edge, the corresponding lateral margin of the recess, arranged in the wall of the instrument shaft and cooperating with the cutting edge, can also be beveled or sharpened.

The cutting edge is preferably arranged on the actuating rod in such a way that it travels past the recess when the actuating rod is moved in order to open the needle receiver. The cutting edge can more preferably be set back in the proximal direction to such an extent that, in order to obtain the cutting action, the actuating rod has to be moved farther in the distal direction than is necessary for normally opening the receiving gap between the two clamping surfaces. That is to say, in order to sever a thread, the handle has to be pressed together more than is required for opening the needle receiver. In this way, a precise control of the individual functions of the instrument is possible.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is an overall longitudinal side view of a needle holder according to and embodiment of the invention;

FIG. 2 is a longitudinal detail view, partially in section, of the distal end of the needle holder according to FIG. 1;

FIG. 3 is an enlarged view of the distal end from the sectional detail according to FIG. 2; and

FIG. 4 is a transverse sectional view along the line IV-IV in FIG. 3.

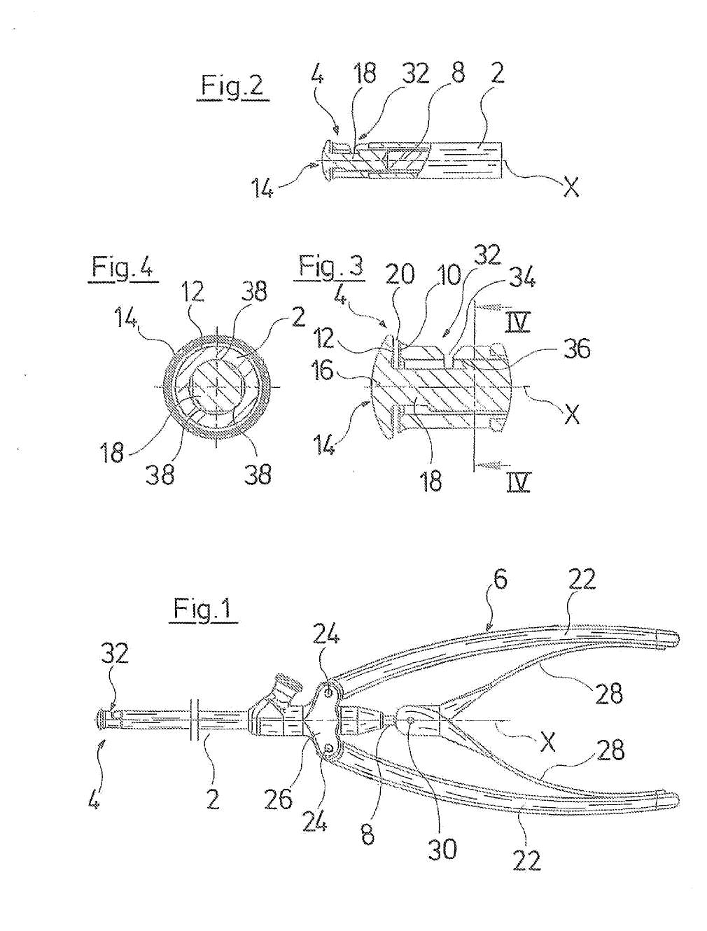

### DETAILED DESCRIPTION OF THE INVENTION

The needle holder according to the invention has an instrument shaft 2, at the distal end of which a needle receiver 4 is arranged and at the proximal end of which a handle 6 is arranged. In the interior of the instrument shaft 2, an actuating rod 8 extends in the axial direction, i.e. along the longitudinal axis X of the instrument.

The needle receiver 4 at the distal end of the instrument is formed by two clamping surfaces 10 and 12, between which needles can be clamped. A first clamping surface 10 is formed directly on the distal end of the instrument shaft 2. This clamping surface 10 is formed by the distal end face of the wall of the instrument shaft 2. For this purpose, the wall of the instrument shaft 2 projects radially outward at the distal end in order to create an enlarged clamping surface 10.

The second clamping surface 12 lies opposite the first clamping surface 10 and is formed on a mushroom-shaped clamping element 14. The first clamping surface and the second clamping surface 12 are ring-shaped and concentric to the longitudinal axis X of the instrument and they extend perpendicularly, i.e. at an angle of 90 degrees to the longitudinal axis of the instrument.

The clamping element 14 is composed of a disk-shaped part 16, which has a substantially circular outer contour and likewise extends concentrically to the longitudinal axis X of the instrument. The second clamping surface 12 is formed on that surface of the disk-shaped part 16 facing toward the proximal end of the instrument and extending perpendicular to the longitudinal axis X of the instrument. Moreover, starting from this side, a foot or pin 18, which is designed in one piece with the disk-shaped part 16, extends in the proximal direction. This pin 18 provides the connection to the actuating rod 8 and is connected to the distal end of the actuating rod 8. It would alternatively be conceivable for the pin 18 to be formed in one piece with the actuating rod. The second clamping surface 12 surrounds the pin 18 in a ring shape and extends radially outward from the pin 18.

By virtue of the fact that the clamping element 14 is fixedly connected to the actuating rod 8, the clamping element 14 is likewise movable in the axial direction X by axial movement of the actuating rod 8. In this way, the clamping surfaces 10 and 12 can be moved toward each other, in order to close the clamping gap or gap 20 between the clamping surfaces 10 and 12. When the actuating rod 8 is moved in the distal direction, the clamping element 14 also moves in the distal direction, and the clamping surfaces 10 and 12 move away from each other, such that a gap 20 is formed, or the gap 20 is enlarged, between the clamping surfaces 10 and 12. The gap 20 is likewise ring-shaped and open toward its outer circumference. Since this clamping gap 20 annularly surrounds the longitudinal axis X of the instrument, needles can be received in this gap from all sides of the longitudinal axis X. The needle holder is thus made easier to handle, since it no longer has to be oriented in a specific receiving position relative to the needle. Instead, there are now a plurality of receiving positions.

In order to improve the clamping of needles between the clamping surfaces 10 and 12, the latter are structured with staggered teeth such that, in addition to a needle being clamped with a force fit, a certain degree of form fit is also provided, which avoids the needle slipping out of the gap 20 in the radial direction.

The secure clamping in the clamping gap 20 is ensured by the special design of the handle 6, which contains restoring springs, said restoring springs ensuring that the actuating rod 8 and therefore the clamping element 14 are at all times subject to a force in the proximal direction, such that the two clamping surfaces 10 and 12 are kept bearing on each other by application of force in the rest position. In this way, a secure clamping of a needle in the gap 20 is ensured in the rest position without the need for additional locking elements.

The handle 6 comprises two grip members 22, which are arranged symmetrically with respect to the longitudinal axis X of the instrument. The grip members 22 are each mounted in an articulated manner at a hinge point 24 on a grip holder 26 formed on the proximal end of the instrument shaft 2. That is to say, they are pivotable here about a pivot axis extending perpendicular to the longitudinal axis X of the instrument, wherein the two pivot axes extending parallel to each other at the two hinge points 24. The two hinge points 24 are spaced apart from each other in a direction transverse to the longitudinal axis X of the instrument and to the pivot axes.

At their proximal end, the two grip members 22 are each connected to a leaf spring element 28. The two leaf spring elements 28 act as the above-described restoring spring. The connection between the grip members 22 and the leaf spring elements 28 is in each case fixed, i.e. not articulated. The leaf spring elements 28 are each curved in such a way that, as they extend in the distal direction, they move away from the inner surfaces, i.e. the mutually facing inner surfaces, of the grip members 22, with the result that the two leaf spring elements 28 approach each other along their extent in the distal direction and meet at a common point of attachment 30. At this point of attachment 30, the leaf spring elements 28 are connected in an articulated manner to the proximal end of the actuating rod 8. The articulated connection here signifies a pivotable arrangement about a pivot axis extending perpendicular to the longitudinal axis X of the instrument and parallel to the pivot axes at the hinge points 24. The point of attachment 30 lies on the longitudinal axis X of the instrument, i.e. in the plane of symmetry of the handle, and is spaced apart proximally from the hinge points 24 in the direction of the longitudinal axis X of the instrument.

In order to open the needle receiver 4, i.e. in order to move the clamping element 14 in the distal direction and move the clamping surfaces 10 and 12 away from each other, the two grip members 22 are pressed together, i.e. moved toward each other. During the resulting pivoting movement of the two grip members 22 about the respective hinge points 24, the proximal ends of the grip members 22 thus approach each other. During this pivoting movement, the two leaf spring elements 28 are elastically deformed and, as a result of the fixed connection at the proximal end, the distal ends of the leaf spring elements 28 shift distally with the point of attachment 30, as a result of which the actuating rod 8 is advanced in the distal direction, such that the clamping element 14 is also shifted distally via the pin 18, and the gap 20 is formed between the clamping surfaces 10 and 12, or the gap 20 is widened. To close the gap 20, the grip members 22 are released, such that they move back to their starting position, i.e. pivot away from each other, on account of the spring action of the leaf spring elements 28. The actuating rod 8 is then pulled back in the proximal direction, as a result of which the clamping element 14 is also moved proximally and the second clamping surface 12 approaches the first clamping surface 10, such that a needle is clamped in the gap 20, or, in the event that there is no needle present in the gap 20, the second clamping surface 12 comes to bear on the first clamping surface 10. By the spring action of the leaf spring elements 28, which tend to force the grip members 22 apart and keep the actuating rod 8 in its proximal end position, a clamping force is generated between the clamping surfaces 10 and 12, which is suitable for clamping a needle securely in the gap 20. Therefore, no additional locking elements are needed.

The needle holder shown also has a thread cutter 32. The latter is arranged near the distal end of the shaft 2 but set back in the proximal direction relative to the needle receiver 4. The thread cutter 32 is formed by a recess in the form of a slit 34 in the wall of the shaft. The slit 34 extends in the tangential direction, or in a chord shape, all the way through the wall of the instrument shaft 2 and into the interior of the instrument shaft 2. The slit 34 extends so deep in the radial direction and so far in the circumferential direction that, between its outer ends, a thread can extend in a chord shape through the interior of the instrument shaft 2. To sever the thread, a cutting edge 36 is formed on the pin 18. This cutting edge 36 slides along the inside wall of the instrument shaft 2 in the area of the gap 34. The cutting edge 36 is arranged in such a way that, when the gap 20 is closed, it is situated proximally behind the gap 34 and, by movement of the actuating rod 8 in the distal direction, can be moved past the gap 34, such that it is located on the distal side of the gap 34. A shearing action is thus achieved between the cutting edge 36 and the margins of the gap 34, which shearing action can be used to cut off or sever a thread that is inserted into the gap 34. In order to ensure this shearing action, the pin 18 in the interior of the instrument shaft 2 is guided substantially free of play. However, as can be seen from FIG. 4, the pin does not bear on the inside wall of the instrument shaft 2 all the way round the circumference. Rather, the pin is not configured with a circular outer circumference, and instead segments of the pin 18 are cut away as seen in cross section, such that this pin 18 bears on the inside wall only in several small circumferential areas 38 distributed about the circumference. In this way, the pin is guided free of play and with low friction.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-13. (canceled)

14. A medical needle holder comprising a handle arranged at a proximal end of the holder and a needle receiver arranged at a distal end of the holder, the needle receiver having first and second clamping surfaces, the clamping surfaces facing toward each other and extending in a direction transverse to a longitudinal axis of the holder, and the clamping surfaces being movable relative to each other, via the handle, in a direction of the longitudinal axis of the holder.

15. The medical needle holder according to claim 14, wherein the first and the second clamping surfaces are ring-shaped, and wherein a gap formed between the clamping surfaces is open toward an outer circumference of the holder.

16. The medical needle holder according to claim 14, wherein the clamping surfaces extend in a circular shape about the longitudinal axis of the holder.

17. The medical needle holder according to claim 14, wherein the first clamping surface is arranged fixedly at a distal end of a shaft of the holder, and the second clamping surface is arranged at a distal end of an actuating rod axially movable relative to the shaft.

18. The medical needle holder according to claim 14, wherein the first clamping surface faces in a distal direction of the holder, and the second clamping surface faces in a proximal direction of the holder.

19. The medical needle holder according to claim 14, wherein the second clamping surface projects radially from a pin extending through an opening in the first clamping surface.

20. The medical needle holder according to claim 14, wherein at least one of the first and second clamping surfaces has a structured surface.

21. The medical needle holder according to claim 20, wherein the structured surface comprises staggered teeth.

22. The medical needle holder according to claim 14, further comprising at least one pre-tensioning element, by which the first and second clamping surfaces are kept bearing on each other by application of a force.

23. The medical needle holder according to claim 22, wherein the pre-tensioning element is arranged such that the first and second clamping surfaces are moved away from each other by actuation of the handle.

24. The medical needle holder according to claim 14, wherein the handle has at least one grip member mounted in an articulated manner at its distal end and connected to a leaf spring element at its proximal end, wherein the leaf spring member, starting from the proximal end of the grip member, extends in a distal direction and, at its distal end away from the grip member, is connected to a proximal end of an actuating rod.

25. The medical needle holder according to claim 24, wherein the proximal end of the actuating rod is spaced apart in a proximal direction from an articulated bearing of the grip member.

26. The medical needle holder according to claim 14, further comprising a thread cutter arranged in an area of the distal end, wherein the thread cutter is actuatable by the handle.

27. The medical needle holder according to claim 26, wherein the thread cutter comprises a recess in a circumference of a shaft of the holder, the recess extending in a radial direction entirely through a wall of the shaft, and wherein a cutting edge of the thread cutter is arranged on an axially movable actuating rod in an interior of the shaft in such a way that, upon movement of the actuating rod, the cutting edge travels past the recess.
